(19) United States
(12) Patent Application Publication
(10) Pub. No.: US 2006/0217740 A1
(43) Pub. Date: Sep. 28, 2006

(54) Tip assembly

(75) Inventor: Ziad R. Ghannoum, Trabuco Canyon, CA (US)

Correspondence Address: ALCON, IP LEGAL, TB4-8, 6201 SOUTH FREEWAY, FORT WORTH, TX, 76134, US

(73) Assignee: ALCON, INC.

(21) Appl. No.: 11/089,893

(22) Filed: Mar. 25, 2005

(51) Int. Cl. A61F 9/00

(52) U.S. Cl. 606/107

(57) ABSTRACT

A quick release tip assembly for a liquefaction surgical handpiece. The tip contains a lumen attached to a hub. The hub has a circumferential flange around its base. The handpiece contains a recess into which the hub fits. The bottom of the recess contains a plurality of movable snaps or catches that as sized to capture the flange on the tip hub. A slidable lever is connected to the snaps so that movement of the level causes the snaps to spread apart, thereby releasing the flange and allowing the tip to be easily removed from the handpiece.

### BACKGROUND OF THE INVENTION

This invention relates generally to the field of cataract surgery and more particularly to a tip assembly for use on a liquefaction handpiece.

The human eye in its simplest terms functions to provide vision by transmitting light through a clear outer portion called the cornea, and focusing the image by way of the lens onto the retina. The quality of the focused image depends on many factors including the size and shape of the eye, and the transparency of the cornea and lens.

When age or disease causes the lens to become less transparent, vision deteriorates because of the diminished light which can be transmitted to the retina. This deficiency in the lens of the eye is medically known as a cataract. An accepted treatment for this condition is surgical removal of the lens and replacement of the lens function by an artificial intraocular lens (IOL).

In the United States, the majority of cataractous lenses are removed by a surgical technique called phacoemulsification. During this procedure, a thin phacoemulsification cutting tip is inserted into the diseased lens and vibrated ultrasonically. The vibrating cutting tip liquifies or emulsifies the lens so that the lens may be aspirated out of the eye. The diseased lens, once removed, is replaced by an artificial lens.

Recently, a new cataract removal technique has been developed that involves the injection of hot (approximately 45° C. to 105° C.) water or saline to liquefy or gellate the hard lens nucleus, thereby making it possible to aspirate the liquefied lens from the eye. Aspiration is conducted with the injection of the heated solution and the introduction of a relatively cool irrigating solution, thereby quickly cooling and removing the heated solution. This technique is more fully described in U.S. Pat. No. 5,616,120 (Andrew, et al.), the entire contents of which is incorporated herein by reference.

Handpiece and tips suitable for practicing this technique are described in U.S. Pat. Nos. 5,989,212, 5,997,499, 6,080,128, 6,110,162, 6,179,805 and 6,206,848 (Sussman, et al.), the entire contents of which being incorporated herein by reference. These patents do not disclose any details on how to connect the tip to the handpiece. Current commercial embodiments of liquefaction handpiece use a threaded connection between the tip and the handpiece.

Therefore, a need continues to exist for a quickly releasable assembly for connecting a tip to a handpiece.

### BRIEF DESCRIPTION OF THE INVENTION

The present invention improves upon the prior art by providing a quick release tip assembly for a liquefaction surgical handpiece. The tip contains a lumen attached to a hub. The hub has a circumferential flange around its base. The handpiece contains a recess into which the hub fits. The bottom of the recess contains a plurality of movable snaps or catches that as sized to capture the flange on the tip hub. A slidable lever is connected to the snaps so that movement of the level causes the snaps to spread apart, thereby releasing the flange and allowing the tip to be easily removed from the handpiece.

Accordingly, one objective of the present invention is to provide a tip assembly for a liquefaction surgical handpiece.

Another objective of the present invention is to provide a quick release tip assembly for a liquefaction surgical handpiece.

These and other advantages and objectives of the present invention will become apparent from the detailed description and claims that follow.

### BRIEF DESCRIPTION OF THE DRAWINGS

FIG. 1 is an exploded partial cross-sectional view of the tip assembly of the present invention.

FIG. 2 is an exploded partial cross-sectional view of the tip assembly of the present invention highlighting the catch release mechanism.

### DETAILED DESCRIPTION OF THE INVENTION

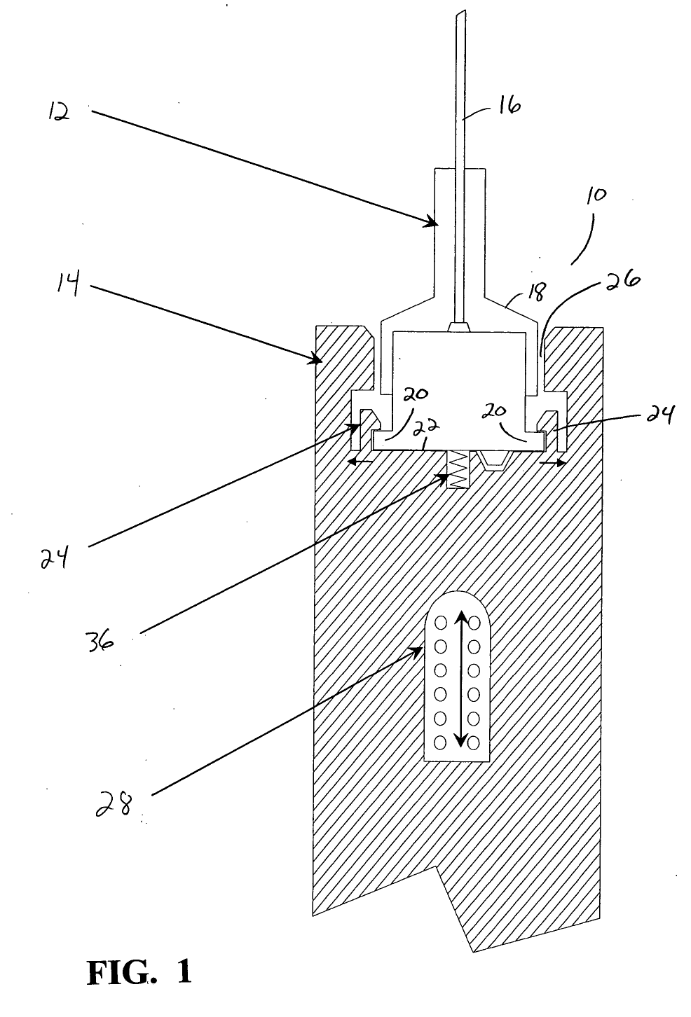

As best seen in FIG. 1, tip assembly 10 of the present invention generally includes tip 12 and handpiece 14. Tip 12 generally includes coaxial cannulae 16 that are connected to hub 18. Hub 18 includes a circumferential flange 20 about proximal base 22. Tip 12 may be made using conventional materials and techniques well-known in the art. Handpiece 14 is of conventional construction and contains recess 26, the bottom of which contains a plurality of movable snaps or catches 24. Recess 26 is sized and shaped to receive proximal base 22 of hub 18 so that catches 24 capture and hold flange 20 when hub 18 is inserted into recess 26.

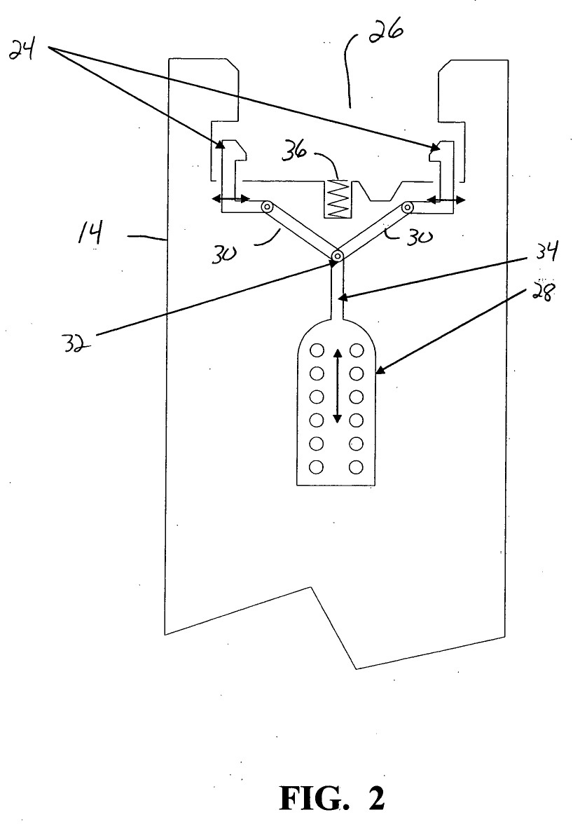

As best seen in FIG. 2, catches 24 are connected internally within handpiece 14 to slidable lever 28 through rods 30, rotatable pin joints 32 and shaft 34. Shaft 34 and lever 28 may be made in a single piece, thereby constraining movement to only one direction. As shown by the arrows, vertical movement of lever 28 causes horizontal movement of catches 24. Spring 36 may also be located in recess 26 so that when hub 18 is nested within recess 26, spring 36 presses against base 22. Such biasing of spring 36 against base 22 assists in releasing hub 18 from catches 24 when catches 24 are pushed away from flange 20 by lever 28.

This description is given for purposes of illustration and explanation. It will be apparent to those skilled in the relevant art that changes and modifications may be made to the invention described above without departing from its scope or spirit.

I claim:

1. A tip assembly, comprising:
a) a tip having a hub with a flange;
b) a handpiece having a recess sized and shaped to receive the hub; and
c) a plurality of movable catches within the recess, the catches spaced to capture and hold the flange with the hub is inserted into the recess.

2. The tip assembly of claim 1 wherein the catches are connected to a lever so that movement of the lever causes movement of the catches.

3. The tip assembly of claim 1 further comprising a spring located in the recess pressing against the hub when the hub is inserted into the recess.

4. A tip assembly, comprising:
a) a tip having a hub with a flange;
b) a handpiece having a recess sized and shaped to receive the hub;
c) a spring located in the recess that biases against the hub when the hub is inserted into the recess;
d) a plurality of movable catches within the recess, the catches spaced to capture and hold the flange with the hub is inserted into the recess; and
e) a lever connected to the catches so that movement of the lever causes movement of the catches.
